(19) United States
(12) Patent Application Publication
(10) Pub. No.: US 2010/0082007 A1
(43) Pub. Date: Apr. 1, 2010

(54) POST-OPERATIVE GARMENTS AND METHODS FOR CONTROLLING POST-OPERATIVE LEAKAGE

(76) Inventor: Brenda Bobo, West Bloomfield, MI (US)

Correspondence Address: O''BRIEN JONES, PLLC, 1951 Kidwell Drive, Suite 550 B, Tyson''s Corner, VA, 22182, US

(21) Appl. No.: 12/241,663

(22) Filed: Sep. 30, 2008

(51) Int. Cl. A61F 13/15

(52) U.S. Cl. 604/385.03; 604/391

(57) ABSTRACT

A post-operative garment comprising a garment body having an outside surface and an inside surface, the garment body being positionable on a body portion of an individual to exert a substantially uniform magnitude of compression pressure upon the body portion; and at least one fastener part on an inside surface of the garment body, the at least one fastener part being configured to engage with a cooperating fastener part on at least one absorbent pad to releasably retain the at least one absorbent pad.

### TECHNICAL FIELD

The present teachings relate generally to post-operative garments. In particular, the present teachings relate to post-liposuction garments and methods for controlling post-operative leakage associated with tumescent liposuction.

### BACKGROUND

The section headings used herein are for organizational purposes only and are not to be construed as limiting the subject matter described in any way.

Because it has eliminated the common problem of surgical bleeding associated with older techniques, the tumescent technique has become the worldwide standard of care for liposuction. The tumescent technique, however, uses a large amount of a dilute tumescent solution that generally remains trapped under a patient's skin after the liposuction procedure. To both improve patient comfort and shorten a patient's recovery time, it is advantageous to quickly drain the trapped tumescent fluid.

Open drainage with bimodal compression is a common technique used after tumescent liposuction to maximize the drainage of the blood-tinged tumescent solution. Open drainage employs numerous tiny round holes made by skin-biopsy punch, otherwise referred to as a tumescent wound, and allows the holes to remain open instead of being closed with sutures. Bimodal compression refers to the sequential use of two different stages of post-liposuction compression: a first stage, wherein a high degree of compression is maintained to maximize the rate of open drainage, and a second stage employing either moderate compression or no compression at all once drainage has ceased.

A patient, therefore, experiences an enormous amount of blood-tinged tumescent solution drainage immediately after the tumescent liposuction procedure. In order to control this drainage, patients must apply various types of absorbent materials and/or pads to their leaking tumescent wounds. The amount of drainage further requires that a patient change absorbent pads frequently during the first few days after the procedure. The conventional process of removing and re-applying a new absorbent pad can be prolonged, painful, and quite difficult for a patient to do without assistance. For example, in some conventional approaches, large flat pads and/or smaller individual pads (e.g. maxi pads) are taped or adhered directly onto the patient's skin. In order to change pads, the patient is required to remove the elasticized wrap or garment used for post-liposuction compression, remove the tape securing each old pad, manually hold each new pad in place over the leaking tumescent wound while securing it with medical tape, and finally re-apply the elasticized wrapping or compression garment.

Post-liposuction garments also have been developed that contain, for example, rows of inside pockets to secure retail maxi pads and/or medical pads into the garment itself. Such garments can act like absorption pad holding systems, and provide a simplified system for a patient to apply and remove pads over the above-described direct application approach.

Issues that can arise, however, in utilizing these types of post-liposuction garments include the need to also provide an effective amount of compression pressure. In this regard, it would be desirable for a post-liposuction garment to not only accommodate painless and straightforward absorbent pad application and removal, but also to provide a relatively high degree of compression pressure to the tumescent wound. It also may be desirable to provide a garment that is relatively easy for a patient to put on and to remove without assistance. In addition, it may be desirable to provide a garment that is configured to easily accept and firmly secure absorbent pads, so that the pads are unable to shift and move against a tumescent wound, which can aggravate the wound. It also may be desirable to provide a garment that facilitates leakage control and distributes a compressive force relatively uniformly across the tumescent wound.

### SUMMARY

The present teachings may solve one or more of the above-mentioned problems and/or may demonstrate one or more of the above-mentioned desirable features. Other features and/or advantages may become apparent from the description that follows.

In accordance with various exemplary embodiments, the present teachings provide a post-operative garment comprising a garment body having an outside surface and an inside surface, the garment body being positionable on a body portion of an individual to exert a substantially uniform magnitude of compression pressure upon the body portion, and at least one fastener part on an inside surface of the garment body, the at least one fastener part being configured to engage with a cooperating fastener part on at least one absorbent pad to releasably retain the at least one absorbent pad.

In accordance with various additional exemplary embodiments, the present teachings provide a method for controlling post-operative leakage comprising releasably fastening at least one absorbent pad to a surface of a garment body; and positioning the garment body on a body portion so as to situate the at least one absorbent pad against a leaking wound and so as to provide a substantially uniform compressive pressure to the body portion.

Additional objects and advantages will be set forth in part in the description which follows, and in part will be obvious from the description, or may be learned by practice of the present teachings. The objects and advantages may be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

The accompanying drawings, which are incorporated in and constitute a part of this specification, illustrate exemplary embodiments and together with the description, serve to explain certain principles. In the drawings:

FIG. 1 is an perspective view of a post-liposuction waist garment worn around the midsection of a body in accordance with an exemplary embodiment of the present teachings;

FIG. 2 is an inside, isometric perspective view of the garment of FIG. 1;

FIG. 3 is a enlarged view of the absorbent pad of FIG. 2:

FIG. 4 is a perspective view of a post-liposuction pant garment in accordance with an exemplary embodiment of the present teachings;

FIG. 5 is a perspective view of absorbent pads for use with the garment of FIG. 4;

FIG. 6 is a perspective view of a post-liposuction body-suit garment in accordance with an exemplary embodiment of the present teachings;

FIG. 7 is a perspective view of an arm garment worn around the upper arm of a body in accordance with an exemplary embodiment of the present teachings; and

FIG. 8 is a perspective view of a leg garment worn around the thigh of a body in accordance with an exemplary embodiment of the present teachings.

Although the following detailed description makes reference to illustrative embodiments, many alternatives, modifications, and variations thereof will be apparent to those skilled in the art. Accordingly, it is intended that the claimed subject matter be viewed broadly.

### DESCRIPTION OF EXEMPLARY EMBODIMENTS

Reference will now be made in detail to various embodiments, examples of which are illustrated in the accompanying drawings. However, these various exemplary embodiments are not intended to limit the disclosure. To the contrary, the disclosure is intended to cover alternatives, modifications, and equivalents.

The present teachings contemplate post-operative garments and methods for controlling post-operative leakage. In particular, the post-operative garments may be adorned after liposuction and useful for controlling leakage associated with tumescent liposuction. The present teachings contemplate post-operative garments that apply a sufficient and substantially uniform magnitude of compression pressure upon a patient's tumescent wounds. For example, the magnitude and uniformity of compression pressure supplied by post-operative garments in accordance with various exemplary embodiments of the present teachings may be sufficient to maximize the rate of fluid drainage (e.g., from tumescent wounds after a tumescent liposuction procedure) while reducing bruising that could occur if the compression pressure were to extreme and/or unevenly distributed on the wearer's body. In addition, the post-operative garments in accordance with various exemplary embodiments of the present teachings may provide the patient with a painless and straightforward way of changing absorbent pads.

To control post-operative leakage, for example, associated with tumescent liposuction procedures, various exemplary embodiments of the present teachings contemplate a post-operative garment adapted for accepting and releasably retaining absorbent pads on the inside surface of the garment's body via one or more fasteners. Fasteners suitable for use in accordance with various exemplary embodiments may include any available fastening mechanism or structure, such as, for example, hook and loop fasteners (e.g., Velcro™), snaps, buttons, catches, tape, and/or glue. In various exemplary embodiments, a fastener may comprise a first fastener part configured to cooperate with a second fastener part, wherein the first fastener part is positioned on the inside surface of the garment body and the second fastener part is positioned on an absorbent pad. Those having ordinary skill in the art will understand, however, that a fastener may be configured as a single part positioned on either the inside surface of the garment body or the absorbent pad without departing from the scope of the present teachings, as will become apparent from the description below. Those skilled in the art will further understand that the number and configuration of such fasteners may be chosen as desired based on simplicity, efficiency, the number and/or configuration of the absorbent pads desired to be supported and other such factors.

The post-operative garment can be worn or positioned on a patient's body so as to place and hold absorbent pads directly against the patient's leaking tumescent wounds. Depending on the type of liposuction procedure performed and the location of a patient's tumescent wounds, various different styles and/or configurations (e.g., shapes) of garment bodies can be employed. In various exemplary embodiments, the garment body comprises a wearable piece of clothing encircling an appropriate anatomical portion of a patient's body, such as, for example, pants, shorts, a body suit, a long sleeve shirt, a short sleeve shirt, and/or a tank top. In various additional exemplary embodiments, the garment body comprises an elongated wrap that can be encircled around a particular portion of the patient's body, such as, for example, a torso, leg, or arm. Those having ordinary skill in the art will understand that the present teachings consider a variety of additional styles and/or configurations (e.g., shapes) of garment bodies to accommodate a broad range of absorbent pad configurations, individual body types, and/or body parts.

Those skilled in the art will further understand that the present teachings contemplate garments of various sizes and/or adjustability. By way of example, post-liposuction garments can come in specific sizes, standard size ranges, or one-size fits all sizing, with or without the ability to further adjust the garment to the appropriate size.

When in position, a wearer of the garment may easily secure the garment using one or more closure mechanisms. Closure mechanisms suitable for use in accordance with various exemplary embodiments may include, but are not limited to, for example, zippers, clasps, snaps, buttons, hooks and eyelets, and/or hook and loop fasteners (e.g., Velcro™). By way of example, a closure mechanism may comprise an engaging member and a receiving member, wherein the engaging member is positioned on a first portion of the garment body and the receiving member is positioned on a second part of the garment body. When joined, the engaging member and the receiving member can form a connection between the first portion and the second portion of the garment body. In various exemplary embodiments, the engaging and receiving members may be positioned proximate end portions of the garment body. Those skilled in the art will understand, however, that the closure mechanism may include a variety of different components in a variety of different positions without departing from the scope of the present teachings.

In order to maximize fluid drainage, once secured, the post-operative garment can additionally provide a relatively high degree of compression pressure to the patient's tumescent wounds. By way of example, the garment body can be made out of an expandable, elasticized material that uniformly constricts when secured by the one or more closure mechanisms. Various exemplary embodiments of the present teachings contemplate garment bodies made out of a variety of materials, including but not limited to, for example, spandex, cotton, nylon, and polyester. Those having skill in the art will further understand that different material combinations may be employed to provide not only an appropriate magnitude of compression pressure, but also an acceptable level of patient comfort. Accordingly, various embodiments of the present teachings additionally consider soft, breathable and/or washable garment body materials.

To change absorbent pads, a patient may loosen and/or remove the post-operative garment by opening the one or more closure mechanisms. The patient can subsequently painlessly detach and remove old absorbent pads from the garment body and re-attach new absorbent pads. To control additional tumescent leakage, the patient can reposition and resecure the post-operative garment, for example, over tumescent wounds.

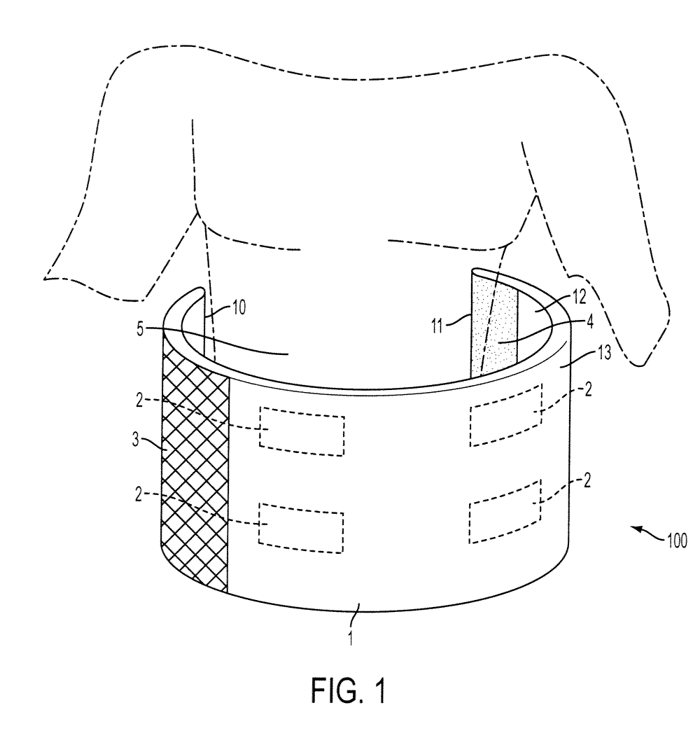

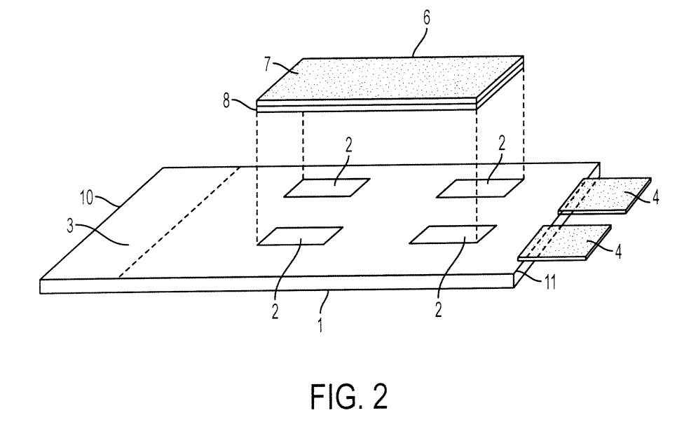

FIGS. 1 and 2 show a post-operative waist garment configured to be worn around the midsection (e.g., torso) of a body in accordance with an exemplary embodiment of the present teachings. FIG. 1 shows a perspective view of the post-operative waist garment in position around a wearer's midsection (e.g., torso) and FIG. 2 shows an isometric perspective view of the inside surface of the waist garment and absorbent pad configured to be received by the waist garment.

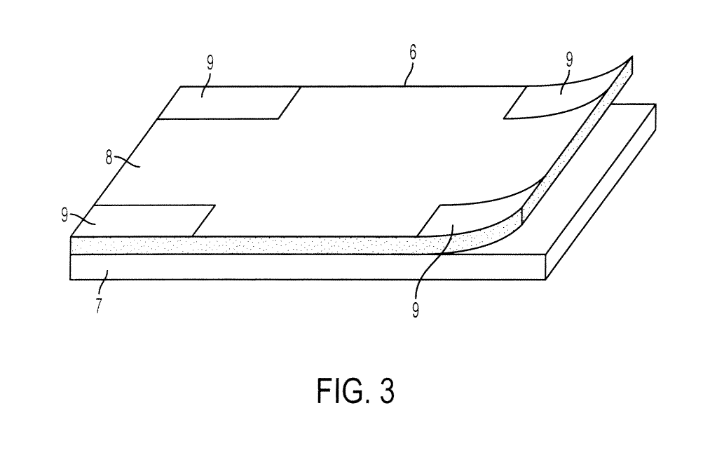

The post-operative waist garment 100 has a garment body 1 dimensioned and configured to generally encircle a torso 5 of a wearer. The garment body 1 is configured to provide a substantially uniform magnitude of compression pressure to the wearer, including the wearer's stomach. For example, the garment body 1 may be elasticized to provide the substantially uniform magnitude of compression. The garment body 1 has an inside surface 12 and an outside surface 13. Fastener parts 2, here shown as the loop portions only of standard hook-and-loop connectors (e.g. Velcro™), are positioned on the inside surface 12 for accepting and releasably retaining an absorbent pad 6 thereon. (See FIG. 2). In an exemplary embodiment, the exterior of the absorbent pad 6 can be provided with the “hook” portions 9 (See FIG. 3), for hook-and-loop retention with loops 2 garment body 1, with which those ordinarily skilled in the art are familiar.

Those having skill in the art would understand that the shape of the post-operative garment 100, here shown as substantially elongated wrap, is schematic and that the garment 100 may have a variety of shapes and configurations without departing from the scope of the present teachings.

The post-operative garment 100 may relatively easily be secured onto the wearer and later loosened and/or removed for absorbent pad replacement utilizing a closure mechanism, such as, for example, an engaging member 3 and a receiving member 4, here shown as cooperating hook-and-loop connectors (e.g. Velcro™). The engaging member 3 and the receiving member 4 can be joined to form a connection between a first end portion 10 of the garment body 1 and a second end portion 11 of the garment body 1. Those having ordinary skill in the art would understand that the positions of engaging member 3 and receiving member 4 on the garment body 1 may be reversed with each other.

It will further be understood by those skilled in the art that the type, number and positioning of the closure mechanisms 3 and 4 depicted in FIG. 1 is exemplary only and non-limiting of the present teachings, and that a variety of types, positions and/or number of closure mechanisms 3 and 4 are envisioned without departing from the scope of the present teachings. Thus, a variety of closure mechanisms commonly used on conventional clothing and garments, including but not limited to, for example, snaps, buttons, zippers, and/or ties, may be employed. In selecting closure mechanisms and/or location of the closure mechanisms on the garment body, those skilled in the art will appreciate that factors such as ease of manipulability, comfort, and/or ease of removal of the garment upon opening of the closure devices may be considered.

FIG. 3 shows an enlarged view of the absorbent pad of FIG. 2 in accordance with an exemplary embodiment of the present teachings. The absorbent pad 6 has an absorption layer 7 and an impermeable layer 8. The absorption layer 7 is adapted for positioning against a tumescent wound, and can be made out of any material that will adequately absorb fluid drainage into the absorbent pad 6. The impermeable layer 8 is adapted for positioning against the inside surface of the garment body 100, and can be made out of any material that will adequately prevent fluid leakage out of the absorbent pad 6. Those having ordinary skill in the art would understand how to select the material, thickness and size of layers 7 and 8 as desired and based on various factors, such as, for example, absorption and resistance capabilities, comfort, hygiene, cost-effectiveness, the size and location of the tumescent wounds, etc. Various embodiments of the present teachings contemplate using a unique post-Iiposuction medical pad for the absorbent pad 6, whereas various additional embodiments consider utilizing conventional super absorbent pads, such as for example, retail maxi pads.

As mentioned above, The impermeable layer 8 of the absorbent pad 6 can be provided with “hook” portions 9 for traditional hook-and-loop retention and mating with loop portions 2 on garment body 1. In various embodiments of the present teachings, the inside surface 12 of the garment body 100 can instead function as a fastener part by being made of a fluff-type fabric that attaches with the hook portions 9. Conversely, various additional embodiments of the present teachings contemplate fastener parts 2 being hook portions, and the impermeable surface 8 being provided with loop portions 9, or consisting of the fluff-type fabric.

It will be understood by skilled artisans of course that the number and positioning of the fastener parts 2 and 9 depicted in FIGS. 1 through 3 and the absorbent pad 6 depicted in FIGS. 2 and 3 are exemplary only and non-limiting of the present teachings, and that a variety of types, positions, and/or number of fastener parts 2 and 9 and absorbent pads 6 are envisioned without departing from the scope of the present teachings. By way of example only, it is envisioned that absorbent pad 6 may include fasteners 9 in the form of adhesive sufficient to secure the absorbent pad 6 to the inside surface 12 of the garment body 1 while also permitting the absorbent pad 6 to be relatively easily removed, for example without leaving adhesive residue on the inside surface 12. Those having ordinary skill in the art are familiar with such adhesive fasteners. Such adhesive fasteners may be provided with a removable lining (not shown), for example, made of plastic, in order to protect the adhesive prior to adhering the absorbent pad 6 to the garment body 1; those skilled in the art are readily familiar with such protective linings used with adhesive fasteners.

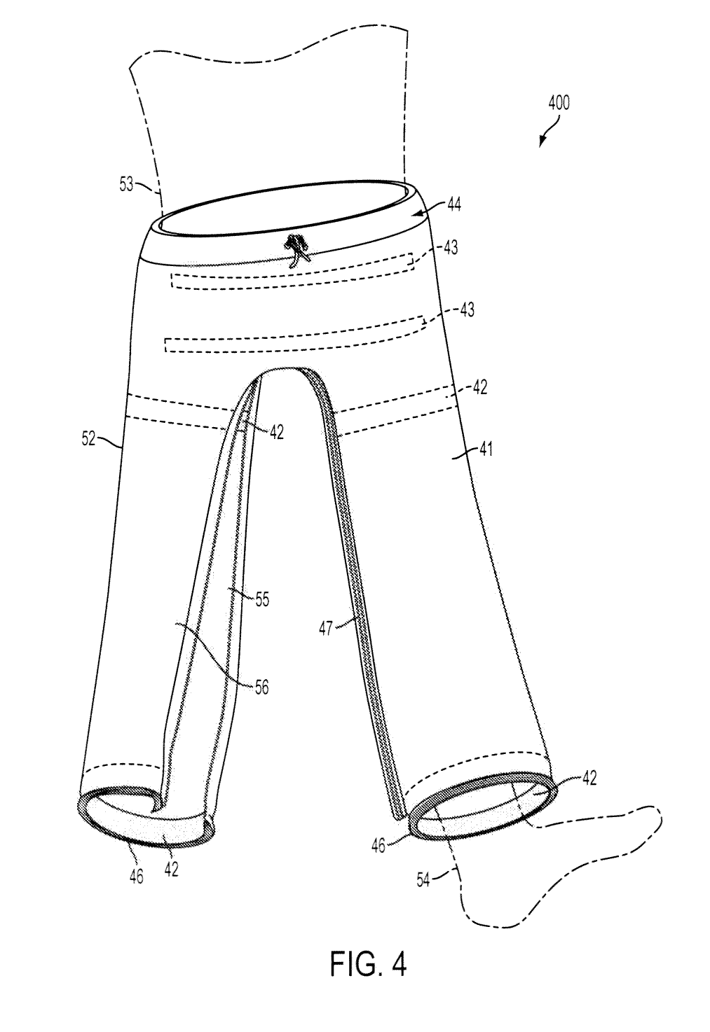

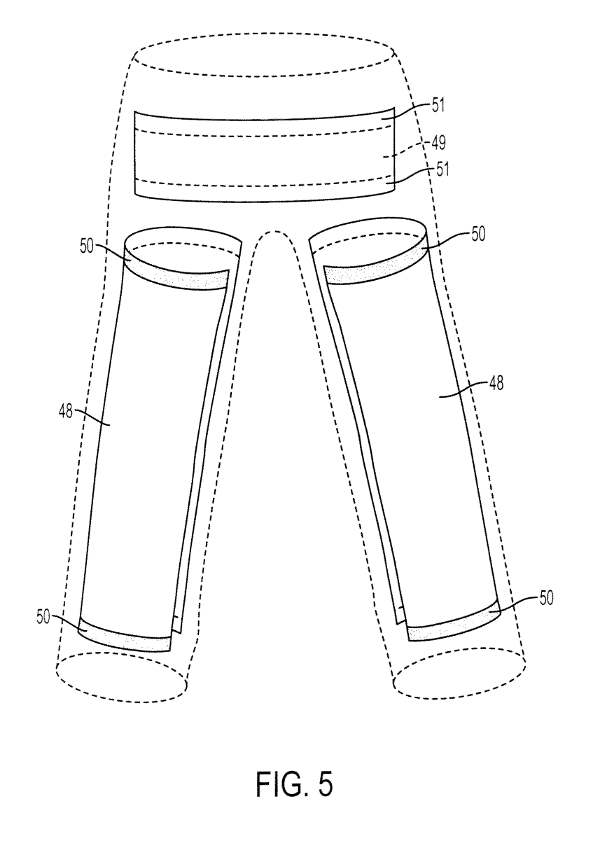

FIGS. 4 and 5 show a perspective view of a post-operative pant garment in accordance with an exemplary embodiment of the present teachings. The post-operative pant garment 400 has a garment body 41 generally encircling an anatomical portion 52 of a wearer between an abdomen area 53 and an ankle area 54. For example, as depicted in FIGS. 4 and 5, the garment body 41 may be configured substantially as pants configured to provide a substantially uniform magnitude of compression pressure to the wearer including the thighs, buttocks, and/or lower abdomen. By way of example, the garment body 41 may be elasticized to provide a substantially uniform compression.

The garment body 41 has an inside surface 55 and an outside surface 56. Fasteners 42 and 43, here shown as the loop portions only of standard hook-and-loop connectors (e.g. Velcro™), are positioned on the inside surface 55 for accepting and releasably retaining absorbent pads 48 and 49 thereon. (See FIG. 5). The exterior of the absorbent pads 48 and 49 can be provided with the “hook” portions 50 and 51, respectively, for hook-and-loop retention, with which those ordinarily skilled in the art are familiar. In various exemplary embodiments of the present teachings, the inside surface 55 of the garment body 41 can be configured to cooperate with a fastener on the absorbent pads 48, 49 by being made of a fluff-type fabric that adheres to the hook portions 50 and 51 on the absorbent pads 48 and 49. Conversely, various additional embodiments of the present teachings contemplate fastener parts 42 and 43 being hook portions only, and the exterior of the absorbent pads 48 and 49 being provided with loop portions 50 and 51, or consisting of the fluff-type fabric.

It will be understood by ordinarily skilled artisans of course that the number and positioning of the fasteners 42 and 43 depicted in FIG. 4 and the absorbent pads 48 and 49 depicted in FIG. 5 are exemplary only and non-limiting of the present teachings, and that a variety of types, positions, and/or number of fasteners 42 and 43 and absorbent pads 48 and 49 are envisioned without departing from the scope of the present teachings.

The post-operative garment 400 may be secured onto the wearer and later loosened and/or removed for absorbent pad replacement using closure mechanisms 44 and 47, here shown as a draw string (which may be elastic) and a zipper, respectively. It will further be understood by those skilled in the art that the type, number, and/or positioning of the closure mechanisms 44 and 47 depicted in FIG. 4 are exemplary only and non-limiting of the present teachings, and that a variety of types, positions and/or number of closure mechanisms 44 and 47 are envisioned without departing from the scope of the present teachings.

In order to capture excess drainage, various exemplary embodiments of the present teachings further contemplate the post-operative garment 400 including elastic bands 46. The elastic bands 46 may supply added compression pressure at additional garment body 41 openings (e.g. the openings of the legs of the pants garment body 41 in the exemplary embodiment of FIGS. 4 and 5) to provide additional leakage control by helping to prevent or hinder leakage out of those openings.

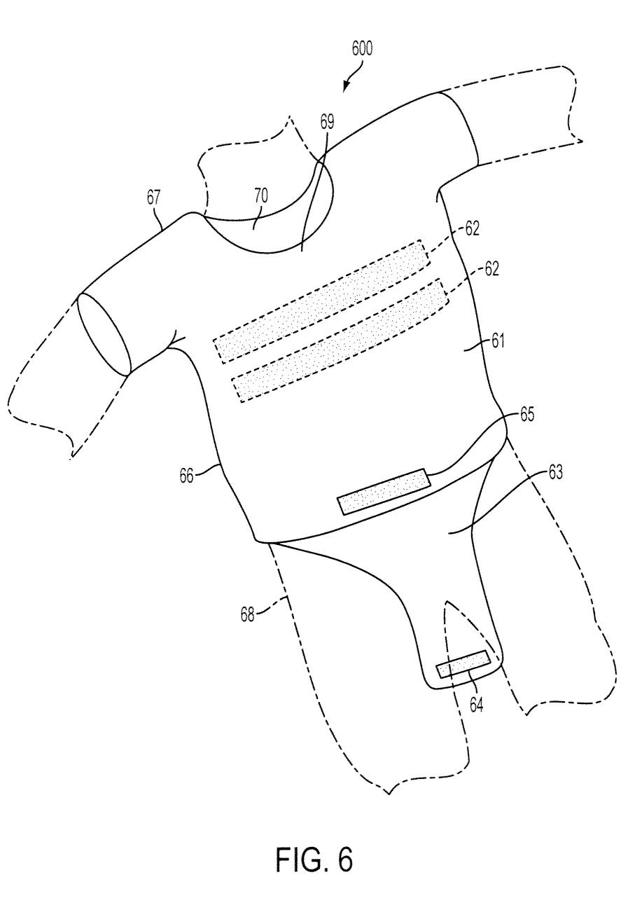

FIG. 6 shows a perspective view of a post-operative body-suit garment in accordance with an exemplary embodiment of the present teachings. A post-operative garment 600 has a garment body 61 generally encircling an anatomical portion 66 of a wearer between a shoulder area 67 and an upper thigh area 68. The garment body 61 is configured to provide a substantially uniform magnitude of compression pressure to the wearer including, for example, the breasts and stomach. The garment body 61 may be elasticized to provide the substantially uniform magnitude of compression. The garment body 61 has an inside surface 69 and an outside surface 70. Fastener parts 62, here shown as the loop portions of standard hook-and-loop connectors (e.g. Velcro™), are positioned on the inside surface 69 for accepting and releasably retaining absorbent pads thereon. As above, in various embodiments of the present teachings, the inside surface 69 of the garment body 600 can instead function as a fastener part by comprising a flufftype fabric that adheres to the hook portions of Velcro™ attached to the absorbent pads, or conversely fastener parts 62 can comprise hook portions only and the absorbent pads may include a fluff-type fabric configured to engage with the hook portions. Those skilled in the art will similarly understand that the type, number and/or positioning of the fastener parts 62 depicted in FIG. 6 are exemplary only and non-limiting of the present teachings, and that a variety of types, positions and/or number of fastener parts 62 are envisioned without departing from the scope of the present teachings.

The post-operative garment 600 may be secured onto the wearer and later loosened and/or removed for absorbent pad replacement utilizing crotch piece 63, here shown in an open position. The crotch piece 63 is conventionally securable via a closure mechanism, here shown as cooperating hook-and-loop connectors 64 and 65 (e.g. Velcro™). By way of example, the crotch piece 63 can be wrapped between a patient's inner thighs and secured to the front of the garment body 61 via connectors 64 and 65. It will further be understood by those skilled in the art that the type, number and positioning of the closure mechanisms 64 and 65 depicted in FIG. 6 are exemplary only and non-limiting of the present teachings, and that a variety of types, positions and/or number of closure mechanisms 64 and 65 are envisioned without departing from the scope of the present teachings.

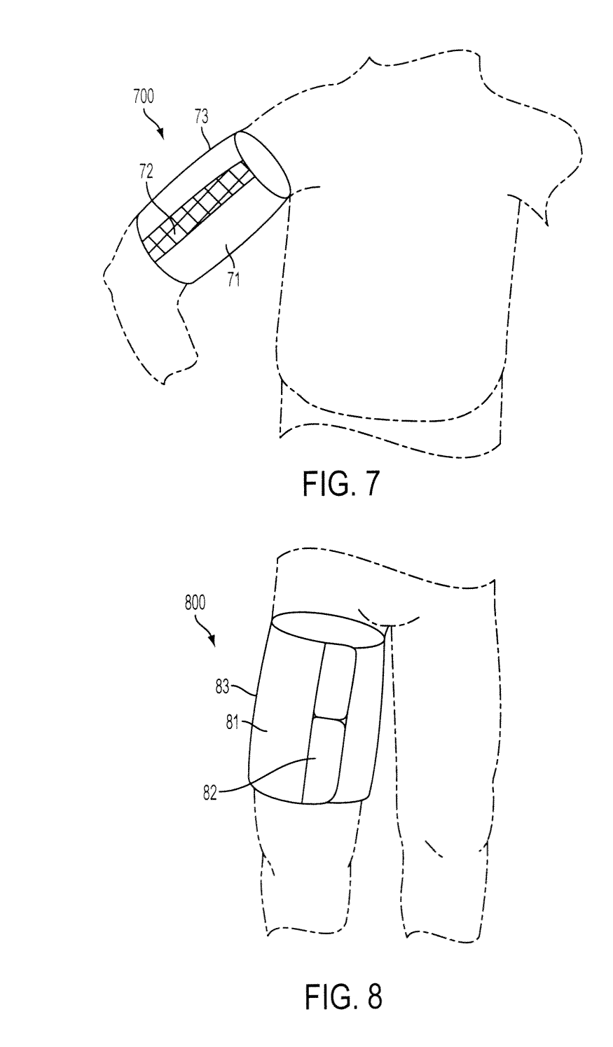

FIGS. 7 and 8 show respective perspective views of an arm garment and a leg garment in accordance with exemplary embodiments of the present teachings. Post-operative garment 700 has a garment body 71 dimensioned and configured to generally encircle an upper arm 73 of a wearer. The garment body 71 is configured, for example by being elasticized, to provide a substantially uniform magnitude of compression pressure to the wearer including the arm. The post-operative garment 700 may relatively easily be secured onto the wearer and later loosened and/or removed for absorbent pad replacement utilizing closure mechanism 72. Post-operative garment 800 has a garment body 81 dimensioned and configured to generally encircle a thigh 83 of a wearer. The garment body 81 is configured to provide a substantially uniform magnitude of compression pressure, for example by being elasticized, to the wearer including the leg. The post-operative garment 800 may easily be secured onto the wearer and later loosened and/or removed for absorbent pad replacement utilizing closure mechanism 82.

Those having skill in the art would understand that the shape of the post-operative garments 700 and 800, here shown as substantially elongated wraps, is schematic and that the post-liposuction garments may have a variety of shapes and configurations. It will further be understood by those skilled in the art that the type, number and positioning of the closure mechanisms 72 and 82 depicted in FIGS. 7 and 8 are exemplary only and non-limiting of the present teachings, and that a variety of types, positions and/or number of closure mechanisms 72 and 82 are envisioned without departing from the scope of the present teachings.

While the present teachings have been disclosed in terms of exemplary embodiments in order to facilitate better understanding of the invention, it should be appreciated that the invention can be embodied in various ways without departing from the scope of the present teachings. Accordingly, while the present teachings have been disclosed mostly in terms of post-liposuction care, the teachings as disclosed work equally well for controlling postoperative leakage associated with other procedures. Further, while various exemplary embodiments have described the use of hook and loop type fastener mechanisms to secure the absorbent pads to the garment bodies, such fasteners are exemplary and non-limiting and those having skill in the art would understand that various other types of fastener mechanisms may be utilized and have been described above. Therefore, the invention should be understood to include all possible embodiments which can be embodied without departing from the scope set out in the appended claims.

Those ordinarily skilled in the art will appreciate that at least some of the features disclosed with respect to one or more exemplary embodiments may also be used in combination with other exemplary embodiments even if not explicitly described. Those with ordinary skill in the art would understand from the present teachings how to modify an exemplary embodiment to incorporate a feature that may be set forth in another exemplary embodiment without departing from the scope of the present teachings.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the written description and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” include plural referents unless expressly and unequivocally limited to one referent. Thus, for example, reference to “a garment body” includes two or more different garment bodies. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the system and method of the present disclosure without departing from the scope its teachings. Other embodiments of the disclosure will be apparent to those skilled in the art from consideration of the specification and practice of the teachings disclosed herein. It is intended that the specification and embodiment described herein be considered as exemplary only.

What is claimed is:

1. A post-operative garment comprising:
a garment body having an outside surface and an inside surface, the garment body being positionable on a body portion of an individual to exert a substantially uniform magnitude of compression pressure upon the body portion; and
at least one fastener part on an inside surface of the garment body, the at least one fastener part being configured to engage with a cooperating fastener part on at least one absorbent pad to releasably retain the at least one absorbent pad.

2. The post-operative garment of claim 1, further comprising at least one closure mechanism configured to secure the garment body on the body portion.

3. The post-operative garment of claim 1, wherein the garment body is configured to situate the at least one absorbent pad against a tumescent wound on the body portion when the at least one absorbent pad is releasably retained by the garment body.

4. The post-operative garment of claim 3, wherein the garment body is configured to exert a magnitude of compression pressure sufficient to drain the tumescent wound.

5. The post-operative garment of claim 1, wherein the garment body comprises an elasticized material.

6. The post-operative garment of claim 1, wherein the garment body comprises a wearable piece of clothing.

7. The post-operative garment of claim 6, wherein the wearable piece of clothing comprises a pair of pants.

8. The post-operative garment of claim 6, wherein the wearable piece of clothing comprises a body suit.

9. The post-operative garment of claim 1, wherein the garment body comprises an elongated wrap.

10. The post-operative garment of claim 9, wherein the elongated wrap is configured to generally encircle a torso of the wearer.

11. The post-operative garment of claim 9, wherein the elongated wrap is configured to generally encircle an arm of the wearer.

12. The post-operative garment of claim 9, wherein the elongated wrap is configured to generally encircle a thigh of the wearer.

13. The post-operative garment of claim 1, wherein the at least one fastener part comprises a hook-and-loop connecter.

14. The post-operative garment of claim 1, wherein the at least one closure mechanism comprises at least one engaging member positioned on a first end portion of the garment body and at least one receiving member positioned on a second end portion of the garment body, the engaging member and the receiving member being configured to be engaged with one another to form a connection between the first end portion and the second end portion of the garment body.

15. The post-operative garment of claim 1, wherein the at least one closure mechanism is a zipper.

16. The post-operative garment of claim 1, wherein the at least one closure mechanism is Velcro™.

17. A method for controlling post-operative leakage comprising:
releasably fastening at least one absorbent pad to a surface of a garment body; and
positioning the garment body on a body portion so as to situate the at least one absorbent pad against a leaking wound and so as to provide a substantially uniform compressive pressure to the body portion.

18. The method of claim 17, wherein the at least one absorbent pad comprises an absorption layer and an impermeable layer, and wherein positioning the garment body comprises positioning the garment body so as to situate the absorption layer of the at least one absorbent pad against the leaking wound.

19. The method of claim 17, wherein the at least one absorbent pad comprises an absorption layer and an impermeable layer, and wherein releasably fastening the at least one absorbent pad to a surface of the garment body comprises positioning the impermeable layer against an inside surface of the garment body.

20. The method of claim 17, wherein releasably fastening the absorbent pad comprises releasably fastening the absorbent pad to an inside surface of the garment body.

21. The method of claim 17, wherein releasably fastening the absorbent pad comprises releasably engaging a first fastening part on the absorbent pad with a second fastening part on the garment body.

22. The method of claim 17, further comprising securing the garment body onto the body portion with at least one closure mechanism.

23. The method of claim 17, wherein positioning the garment body on a body portion so as to situate the at least one absorbent pad against a leaking wound comprises positioning the garment body on a body portion so as to situate the at least one absorbent pad against a tumescent wound.
